(19) United States
(12) Patent Application Publication
(10) Pub. No.: US 2020/0170928 A1
(43) Pub. Date: Jun. 4, 2020

(54) NATURAL STYLING GEL WITH CARRAGEENAN AND A FILM FORMER

(71) Applicant: Henkel AG & Co. KGaA, Duesseldorf (DE)

(72) Inventors: SANDRA BRANDT, Pinneberg (DE); ANNA PULS, Winsen (Luhe) (DE)

(73) Assignee: Henkel AG & Co. KGaA, Duesseldorf (DE)

(21) Appl. No.: 16/680,896

(22) Filed: Nov. 12, 2019

(30) Foreign Application Priority Data
Dec. 4, 2018 (DE) 10 2018 130 893.0

(51) Int. Cl. A61K 8/9717; A61K 8/73; A61K 8/362; A61K 8/92; A61Q 5/06

(52) U.S. Cl. CPC A61K 8/9717; A61K 8/732; A61Q 5/06; A61K 8/922; A61K 8/362

(57) ABSTRACT

The present disclosure relates to a cosmetic agent for temporarily reshaping keratin fibres. The cosmetic agent includes, a cosmetic carrier, a film former, and a natural thickener. The natural thickener includes carrageenan and/or salts thereof. The film former includes a hydroxypropyl group-modified starch compound.

### CROSS-REFERENCE TO RELATED APPLICATION

This application claims priority to German Patent Application No. 10 2018 130 893.0, filed Dec. 4, 2018, which is incorporated herein by reference in its entirety.

### TECHNICAL FIELD

The present disclosure relates to cosmetic agents for temporarily reshaping keratin fibres, in particular human hair, in a cosmetic carrier including a film former and a natural thickener.

### BACKGROUND

Cosmetic agents which are used to permanently or temporarily shape hair play an important role in the field of cosmetics. Temporary styling intended to provide a good hold without compromising the healthy appearance of the hair, such as its shine, can be attained for example by hairsprays, hair waxes, hair gels, hair mousses, setting lotions, etc.

The most important property of an agent for temporarily shaping keratin fibres lies in providing the treated fibres with a strongest hold possible in the produced form. If the keratin fibres are human hair, reference is also made to a strong hairstyle hold or a high degree of hold of the cosmetic agent. The hairstyle hold is determined substantially by the type and amount of used polymers.

Numerous cosmetic agents which ensure a temporary styling are known in the prior art. Corresponding agents usually contain synthetic polymers as shaping components. Agents for temporary styling may also often contain synthetic polymers as viscosity-modifying substances. Preparations which contain a dissolved or dispersed polymer may be applied to the hair by employing a propellant gas or by a pump mechanism. Hair gels and hair waxes, by contrast, are generally not applied directly to the hair, but instead are distributed in the hair by employing a comb or using the hands.

Polymer ingredients have different functions in agents for temporary styling. Viscosity-modifying substances formed from polymers may thus be formed. Polyelectrolytes are often used here. Furthermore, the substances that are ultimately responsible for the hairstyle hold constitute polymer components. The synthetic polymers usually used in agents for temporary styling are produced from appropriate synthetically accessible monomers. Said monomers are obtained from fossil substances, for example petroleum, by conversion into the corresponding polymer building blocks, inter alia with expenditure of energy. Since the monomers are already produced synthetically, the polymers usually used are fully synthetic polymers.

Within the scope of a more sustainable handling of raw materials and energy, it is still desirable to use, for cosmetic products, only cosmetic raw materials that are accessible from renewable raw materials with minimal energy consumption. A reduction of the amount, or even replacement of said fully synthetic polymers, however, can be implemented only if the replacement polymers bring about the properties desired for the intended application and allow the keratinous fibres to hold the styled form in a sufficient and stable fashion.

Numerous further properties that the cosmetic agent imparts on the hair must be maintained nevertheless with a reduction of the amount, or a replacement of fully synthetic polymers. Cosmetic agents should thus give the hair volume, and the bounce and silkiness of the keratinous fibres fixed in the style should be maintained. The formation of polymer particles visible to the naked eye on the keratinous fibres must be avoided. Furthermore, the keratinous fibres must not appear lacklustre, but instead should appear to have a natural shine. These and further properties which are imparted to the hair by the cosmetic agent will be referred to hereinafter as styling properties. The development of cosmetic agents which have all desired styling properties in combination still poses difficulties. In particular, this is true for the combination of a strong hold on the one hand and simple, uniform application to the keratinous fibres on the other hand.

Lastly, the cosmetic agent itself should also look nice, so as to promote a decision to purchase it. In the case of styling products, in particular hair gels, the customer usually expects a transparent gel. the replacement of fully synthetic polymers should not result in any disadvantageous properties in respect of the physical appearance of the cosmetic agent.

The problem addressed by the present disclosure lies in providing a cosmetic agent which is suitable for temporarily reshaping keratinous fibres, in which the use of fully synthetic polymers is significantly reduced or in which the use of fully synthetic polymers is spared, wherein the styling properties are retained to a high level, and wherein the cosmetic agent should have an appealing physical appearance.

The problem addressed by the present disclosure is solved by the subject matter of claim 1. A first subject of the present disclosure is therefore a cosmetic agent for temporarily reshaping keratin fibres, in particular human hair, in a cosmetic carrier comprising a film former and a natural thickener, wherein the natural thickener comprises carrageenan and/or salts thereof, and the film former comprises a hydroxypropyl group-modified starch compound.

The cosmetic agent contains carrageenan and/or salts thereof as natural thickener. The term “natural thickener” is intended to mean that the thickener originates from a natural and thus non-synthetic source. Carrageenan is the collective term for a group of long-chain carbohydrates which can be obtained from red algae cells. Carrageenan is constituted by linear, anionic hydrocolloids, which can be differentiated on the basis of their chemical structure and can have different properties. These different types are distinguished by the proportion of galactose and 3,6-anhydrogalactose and by the number of sulfate groups. The sulfate groups can occur in the protonated form as free acid groups, or occur as salts. The thickener is then a carrageenate, carrageen is commercially obtainable.

### BRIEF SUMMARY

Cosmetic agents are provided herein. In an exemplary embodiment, a cosmetic agent for temporarily reshaping keratin fibres includes, a cosmetic carrier, a film former, and a natural thickener. The natural thickener includes carrageenan and/or salts thereof. The film former includes a hydroxypropyl group-modified starch compound.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In accordance with the present disclosure the cosmetic agent comprises a film former. A film former is understood to be constituted by polymers which, as they dry, leave behind a continuous film on the skin, the hair or the nails. Such film formers can be used in a very wide range of cosmetic products, such as face masks, make-up, hair fixers, hairsprays, hair gels, hair waxes, hair conditioners, shampoos or nail varnished. Polymers which have a sufficient solubility in water are preferred. Corresponding solutions which can be applied or further processed in a simple manner can thus be produced.

Within the scope of the present disclosure a film former shall be understood to mean, in particular, polymers which, when used in from about 0.05 to about 20 wt % (in relation to the total weight of the cosmetic agent) aqueous, alcoholic or aqueous-alcoholic solution, are capable of depositing on the hair in the form of a transparent polymer film.

The film former is a hydrophobically modified starch compound, more specifically a hydroxypropyl group-modified starch compound.

It has surprisingly been found that a cosmetic agent which may be substantially free from fully synthetic film formers and which contains a hydroxypropyl group-modified starch compound and also carrageenan may be formulated without suffering any losses in respect of the styling properties.

Starch belongs to the family of homoglycans and is a polycondensation product of D-glucose. Here, starch includes three structurally different polymers of D-glucopyranose, specifically amylose, amylopectin and what is known as an intermediate fraction. Higher plants contain from 0 to about 45 wt % amylose in relation to the dry substance.

The intermediate fraction, which is also referred to as abnormal amylopectin, is situated structurally between the amylose and the amylopectin. The stated amounts for amylopectin defined within the scope of this application comprise the intermediate fraction. Starch is also a reserve carbohydrate which is stored by many plants in the form of starch grains (granules) usually from about 1 to about 200 μm in size in different plant parts, for example in tubers or roots, cereal grains, fruits, and in the pith.

It is preferred if the hydrophobically modified starch compound used as contemplated herein has an amylose content of less than about 25 wt %, in particular of less than about 20 wt %, in each case in relation to the weight of the modified starch compound. It has been found that a starch which contains from about 17 to about 22 wt % amylose and from about 78 to about 83 wt % amylopectin is particularly suitable for achieving the styling properties.

Amylose includes predominantly linear α-1,4-glycosidically linked D-glucose, M_{w} from about 50,000 to about 150,000. The resultant chains form double helices in the starch. Amylopectin, in addition to the α-1,4 links described for amylose, also contains an amount of from about 4 to about 6% of α-1,6 bonds as branching points. The average distance between the branching points is approximately 12 to 17 glucose units. The molar mass of from about 107 to about 7×108 corresponds to approximately 105 glucose units, whereby amylopectin belongs to the largest biopolymers. Said branchings are distributed over the molecule in such a way that a cluster structure with relatively short side chains develops. Two of these side chains each form a double helix. Due to the main branching points, amylopectin is relatively well soluble in water.

Due to a different structure of the carbohydrates, some natural providers of starch are suitable to a greater or less extent. In accordance with a preferred embodiment of the present disclosure, the basis of the hydroxypropyl group-modified starch compound is a starch of plant origin, wherein the starch is preferably obtained from potatoes, maize, rice, peas, acorns, chestnuts, barley, wheat, bananas, millet, sorghum, oats, barley, rye, beans, sweet potato, arrowroot, cassava, tapioca and/or sago, in particular potatoes and/or tapioca. Starches from this origin are of particular importance for the solution to the problem forming the basis of the present disclosure.

A hydroxypropyl group-modified starch compound shall be understood to mean a reaction product from a starch and propylene oxide. Such a reaction product preferably comprises at least one structural unit of formula (PS),

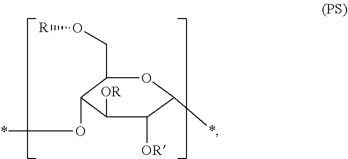

wherein at least one molecular group R, R′ or R″ stands for a group of

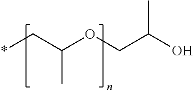

with n≤2, preferably 0, and at most 2 of the molecular groups from R, R′, R″ stand for a hydrogen atom. A bond denoted by the symbol * in formulas of this application corresponds to a free valence of the corresponding structural unit. The hydroxypropyl group-modified starch compound is provided for example by reacting a native starch with propylene oxide. Before the modification with propylene oxide, the starch may have been subjected to various physical or chemical processes, for example a heat treatment, shearing, a thermal acid-hydrolytic, oxidative or enzymatic cleaving, etc.

It is preferred if the hydroxypropyl group-modified starch compound is present in the cosmetic agent not in the form of individual starch grains (granules). For this purpose, the starch grains are opened up, for example by heat or shearing, and the corresponding polysaccharide molecules are released from the composite. The released polysaccharide molecules are modified by using propylene oxide after or prior to release.

Within the scope of a preferred embodiment the hydroxypropyl group-modified starch is gelatinised. If an aqueous suspension of starch is heated or compressed, a tangential swelling of the body with loss of the birefringence, modification of the X-ray structure, and abrupt increase in viscosity of the solution is observed at a critical temperature or pressure. This appearance is referred to as gelatinisation.

The hydroxypropyl group-modified starch compounds are present in the cosmetic agent in a molecular weight distribution. Preferred hydroxypropyl group-modified starch compounds have a mean molecular weight of from about 50 to about 2,500 kDa (weight average). The molecular weight distribution was determined experimentally by employing gel filtration chromatography versus dextran. Particularly preferred cosmetic agents contain hydroxypropyl group-modified starch compounds that have a mean molecular weight (weight average) of from about 100 to about 2,000 kDa, in particular from about 500 to about 1,800 kDa, very particularly preferably from about 700 to about 1,000 kDa.

In order to adjust the molecular weight, the starch is subjected to a mechanical and/or a chemical treatment prior to or after the modification by propylene oxide. To increase the molecular weight, said starch may be cross-linked. A cross-linking of the hydroxypropyl group-modified starch compounds is present when the linear or branched polysaccharide macromolecules of the starch are covalently linked by a crosslinking agent so as to form a three-dimensional, insoluble and only still swellable polymer network. Natural starch is generally un-cross-linked and—if cross-linking were desirable—requires an artificial cross-linking by employing synthesis chemistry. Such an artificial cross-linking may be performed by cross-linking agents. Hydroxypropyl group-modified starch compounds that do not have a cross-linking of this kind are un-cross-linked.

A cross-linking is performed for example by the cross-linking agent epichlorohydrin. To this end, an about 42 wt % mixture of hydroxypropyl group-modified starch compounds is produced in water, the desired amount of epichlorohydrin is stirred into this mixture at room temperature, and, after a stirring time of from about 1 to about 5 hours with control of the viscosity, once the target viscosity has been reached the cross-linked starch is isolated in accordance with conventional methods. It is, however, particularly preferred as contemplated herein if the cosmetic agent, as hydroxypropyl group-modified starch compound, contains at least one un-cross-linked hydroxypropyl group-modified starch compound.

In order to attain a lower molecular weight of from about 100 to about 400 kDa, said starches are preferably exposed to a mechanical cleaving, an enzymatic cleaving (in particular with alpha-amylase, beta-amylase, glucoamylase or debranching enzymes), an acid-hydrolytic cleaving (in particular with hydrochloric acid, sulfuric acid, or phosphoric acid), a thermal cleaving or a reaction with oxidising agents (such as periodate, hypochlorite, chromic acid, permanganate, nitrogen dioxide, hydrogen peroxide or organic percarboxylic acid, preferably with hydrogen peroxide). Kneaders, extruders, stator/rotor machines and/or agitators are suitable for mechanical cleaving.

Oxidative cleavage by hydrogen peroxide is preferred as contemplated herein. For this purpose, the hydroxypropyl group-modified starch compounds may be, for example, added to water, heated to from about 50 to about 70° C., hydrogen peroxide added, and the mixture stirred at from about 70 to about 85° C. for from about 2 to about 5 hours.

The content of propylene oxide in the starch affects the fine tweaking of the hairstyle hold with the hairstyle flexibility and the stability of the cosmetic agent. It has been found that the parameters may be further optimised if the hydroxypropyl group-modified starch compound has a propylene oxide content of from about 1 to about 20 wt %, particularly preferably a propylene oxide content of from about 4 to about 12 wt %, very particularly preferably a propylene oxide content of from about 9.5 to about 10.5 wt %—in relation to the weight of the modified starch. The propylene oxide content can be determined for example by carrying out a Hodges cleaving by employing the method according to DIN EN 13268 (issue date 2007-06).

It has also been found that cosmetic agents as contemplated herein in which the hydroxypropyl group-modified starch compound in an about 43 wt % aqueous solution has a preferred viscosity in the range of from about 150 to about 1,500,000 mPa s (Brookfield viscometer, spindle #7 at about 20° C. and about 20 rpm) are excellently suitable. Particularly suitable hydroxypropyl group-modified starch compounds have viscosities of from about 10,000 to about 200,00 mPa s, particularly preferably from about 25,000 to about 180,000 mPa s (in each case measured under the aforementioned conditions).

The film former of the cosmetic agent in accordance with a preferred embodiment of the present disclosure shall consist “substantially” of the above-mentioned hydrophobically modified starch compounds. Within the scope of the present disclosure, this may preferably also mean that the film formers of the cosmetic agent comprise the above-mentioned hydrophobically modified starch compounds, in particular the hydroxypropyl group-modified starch compound, exclusively, that is to say in an amount of about 100 wt % in relation to the total weight of the film former. The feature that the film former of the cosmetic agent consists “substantially” of the component(s) shall be understood by a person skilled in the art in the sense that further components may be present in the cosmetic agent in a small proportion, for example up to about 1 wt %, preferably up to about 0.5 wt %, in relation to the total weight of the cosmetic agent and may be part of the polymer film without, however, being considered to be film formers in the conventional sense. In other words, the film formed from the film former may also contain further constituents.

In accordance with a preferred embodiment of the present disclosure the cosmetic agent is free from a fully synthetic film former.

The cosmetic agent of the preferred embodiment should comprise substantially no fully synthetically produced polymers used conventionally as film formers. A film former is deemed to be produced fully synthetically within the scope of the present disclosure if all of the monomers constructing the film former are conventionally produced synthetically. Examples of fully synthetically produced film formers include, in particular, copolymers based on acrylate or methacrylate, in particular those that comprise, as monomer constituent, acrylic acid, methacrylic acid, alky(meth)acrylate, hydroxyalkyl(meth)acrylate or maleic acid anhydride; and copolymers which comprise, as monomer constituents. N-vinylpyrrolidone, alkylvinyl ether with alkyl groups comprising 1 to 18 carbon atoms and/or vinyl esters of carboxylic acids with 2 to 18 carbon atoms, in particular polyvinylpyrrolidone/vinyl acetate copolymers; and also polyvinylpyrrolidone, wherein copolymers are understood to mean polymers that include two or more different monomers, and wherein all alkyl groups of the aforesaid monomers can also be branched alkyl groups and/or alkyl groups substituted with functional groups.

Within the scope of the present disclosure the cosmetic agent may comprise up to about 0.05 wt % of fully synthetic polymers which in the conventional sense are film formers, so as to be “substantially” free from fully synthetic film formers. The cosmetic agent may preferably also be free from fully synthetic film formers, i.e. the cosmetic agent comprises 0 wt % of fully synthetic film formers.

Within the scope of the present disclosure, “keratin fibres” shall be understood to mean fur, wool, feathers and in particular human hair.

It has also been found surprisingly that a cosmetic agent which contains a hydroxypropyl group-modified starch compound and carrageenan can be formulated without having to accept any losses in respect of the styling properties, and that the cosmetic agent can be obtained as a transparent gel. A product is thus obtained which has a pleasant physical appearance. In a preferred embodiment of the present disclosure the weight ratio of film former to thickener lies in a range between about 40 to 1 and about 1 to 2, preferably between about 20 to 1 and about 1 to 1, more preferably between about 15 to 1 and about 2 to 1, and in particular is approximately 5 to 1. In this preferred ratio of the components contained mandatorily in the cosmetic agent, the advantageous physical appearance is achieved and the styling properties are particularly advantageous. As a result of these advantageous features, the cosmetic agent is encouraged to assume the form of a transparent mixture,

In accordance with a further preferred embodiment of the present disclosure the cosmetic carrier comprises water. The cosmetic carrier more preferably comprises water which is acidified with a divalent or trivalent organic acid, in particular citric acid. In accordance with alternative, preferred embodiments the organic acid is a carboxylic acid which is even more preferably selected from the group including formic acid, acetic acid, lactic acid, pyrrolidone carboxylic acid, tartaric acid, glycolic acid, nicotinic acid, hydroxyisobutyric acid, hydroxyisovaleric acid or mixtures of these acids. The acid and the natural thickener form an advantageous neutralisation product, which for example can be produced in an aqueous medium by adding chitosan and the corresponding organic carboxylic acids or part of the corresponding organic carboxylic acids.

It has been found that cosmetic agents that comprise film formers comprising a neutralisation product of carrageenan and/or carrageenate with the above acid, in particular with citric acid, and a hydroxypropyl group-modified starch compound bring about styling properties which achieve a high level comparable to that of cosmetic agents comprising fully synthetic film formers,

As contemplated herein it is preferred if the cosmetic agent contains the hydroxypropyl group-modified starch compound in an amount of from about 1.5 wt % to about 20 wt %, preferably from about 2 wt % to about 15 wt %, more preferably from about 3 wt % to about 10 wt %, in each case in relation to the weight of the cosmetic agent.

In accordance with a further preferred embodiment of the present disclosure the cosmetic agent contains

a) from about 70 to about 98 wt %, preferably from about 75 to about 95 wt %, more preferably from about 80 to about 90 wt %, water,
b) from about 1.5 to about 20 wt %, preferably from about 2 to about 15 wt %, more preferably from about 3 to about 10 wt %, hydroxypropyl group-modified starch compound, and
c) from about 0.5 to about 10 wt %, preferably from about 1 to about 7 wt %, more preferably from about 2 to about 5 wt %, carrageenan, in each case in relation to the total weight of the cosmetic agent.

In accordance with a preferred embodiment of the present disclosure the cosmetic agent contains small amounts of a nourishing polymer, which is particularly preferably a cationic homopolymer. Cationic polymers are understood to be polymers which in the main and/or side chain have a group which is “temporarily” or “permanently” cationic. As contemplated herein, “permanently” cationic polymers are those which comprise a cationic group irrespective of the pH value of the agent. These are generally polymers which contain a quaternary nitrogen atom, for example in the form of an ammonium group. Preferred cationic groups are quaternary ammonium groups.

In particular, homopolymers in which the quaternary ammonium groups are bonded via a C1-4 alkylene group to a homopolymer main chain constructed from acrylic acid amide, wherein the three alkyl groups besides the C1-4 alkylene group of the ammonium group are selected independently of one another from C1-10 alkyl groups, have proven to be particularly suitable.

In order to compensate for the positive polymer charge, all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

In accordance with a particularly preferred embodiment of the present disclosure, the cosmetic agent contains, as a nourishing cationic homopolymer, poly(3-acrylamidopropyl)trimethyl ammonium chloride. The nourishing homopolymer is very particularly preferably used in an amount of from about 0.0001 wt % to about 0.2 wt %, preferably in an amount of more than about 0.01 wt % to less than about 0.1 wt %, in relation to the total weight of the cosmetic agent. These nourishing homopolymers fall within the definition of fully synthetic polymers, however they are used in a very small amount. Furthermore, cationic homopolymers based on acrylamide, due to their nature and according to definition, are not film formers in the conventional sense.

In accordance with a further embodiment the cosmetic agent additionally contains emulsifiers. Emulsifiers cause the formation of water-stable or oil-stable adsorption layers at the phase interface, which layers protect the dispersed droplets against coalescence and thus stabilise the emulsion. Emulsifiers are therefore constructed from a hydrophobic and a hydrophilic molecule part, similarly to surfactants. Hydrophilic emulsifiers preferably form O/W emulsions (oil-in-water emulsions) and hydrophobic emulsifiers preferably form W/O emulsions (water-in-oil emulsions). The selection of these emulsifying surfactants or emulsifiers is based here on the substances to be dispersed and the particular outer phase and the fineness of the emulsion.

In accordance with a preferred embodiment of the present disclosure the cosmetic agent also comprises a solubiliser, which is preferably castor oil and/or an alkoxylated castor oil, which more preferably is a castor oil alkoxylated with from about 20 to about 80, even more preferably from about 30 to about 50, ethylene oxide groups, in particular a castor oil alkoxylated with about 40 ethylene oxide groups. In accordance with a further preferred embodiment of the present disclosure the cosmetic agent also contains a solubiliser, in particular a castor oil alkoxylated with about 40 ethylene oxide groups, in an amount of from about 0.05 to about 2 wt %, preferably from about 0.1 to about 1 wt %, more preferably from about 8 to about 12 wt %, in relation to the total weight of the cosmetic agent.

The emulsifiers preferably used offer the advantage of being able to be obtained from natural, non-fully-synthetic raw material sources.

In order to intensify the effects as contemplated herein, the cosmetic agents preferably additionally contain at least one surfactant. Generally, non-ionic, anionic, cationic, and ampholytic surfactants may be contained in cosmetic products. The group of ampholytic or also amphoteric surfactants comprises zwitterionic surfactants and ampholytes. It has proven to be particularly advantageous if the cosmetic agents additionally contain at least one cationic surfactant in order to increase the haircare properties.

Cationic surfactants that can be used with preference are those of the quaternary ammonium compound, esterquat and amidoamine type. Preferred quaternary ammonium compounds are ammonium halides, in particular chlorides and bromides, such as alkyl trimethyl ammonium chlorides, dialkyl dimethyl ammonium chlorides, and trialkyl methyl ammonium chlorides. The long alkyl chains of these surfactants preferably have 10 to 18 carbon atoms, for example as in cetyl trimethyl ammonium chloride, stearyl trimethyl ammonium chloride, distearyl dimethyl ammonium chloride, lauryl dimethylammonium chloride, lauryl dimethyl benzyl ammonium chloride and tricetyl methyl ammonium chloride. In accordance with a preferred embodiment of the present disclosure, alkyltrimethyl ammonium salts are used, more preferably C12-C20 alkyltrimethyl ammonium salts, particularly preferably C16-C18 alkyltrimethyl ammonium chlorides, in particular cetrimonium chloride.

In a preferred embodiment of the present disclosure the cosmetic agent comprises from about 0.01 to about 4 wt %, particularly preferably from about 0.1 to about 3 wt %, and in particular from about 0.2 to about 2 wt % of the cationic surfactant, wherein the amounts stated relate to the total weight of the cosmetic agent.

The application properties of the cosmetic agent may be further increased by the use of small amounts of one or more polyvalent alcohols. Preferred cosmetic agents contain one or more C3 to C12 alkane-1,2 diols, more preferably C3 to C10 alkane-1,2-diols. The cosmetic agent particularly preferably contains propane-1,2-diol and caprylyl glycol. These particularly preferred diols improve the application properties, are nourishing so to speak, and are suitable for forming a stable emulsion together with the particularly preferred emulsifier. In relation to the total weight of the cosmetic agent, the cosmetic agent may contain the alkane-1,2-diol in an amount of from about 0.01 to about 1.5 wt %, more preferably from about 0.02 to about 0.8 wt % in a preferred embodiment of the present disclosure.

In a preferred embodiment of the present disclosure the cosmetic agent contains at least one nourishing oil. Oils are advantageous as nourishing substances since they give the hair a silky shine and make the hair more resistant by being absorbed by the hair. Nourishing oils, however, place higher demands on the product formulation, since they must be incorporated in a stable manner, without demonstrating a disadvantageous reduction or creaming during longer storage periods of product containers.

In accordance with a preferred embodiment of the present disclosure, the cosmetic agent comprises at least one nourishing oil and/or at least one nourishing constituent, more preferably at least one natural nourishing oil, i.e. a substance comprising a triglyceride of natural origin, wherein plant-based nourishing oil(s)/nourishing constituent(s) are particularly preferred. Nourishing constituents are non-polymeric in nature. Plant-based nourishing oils from the group of amaranth seed oil, argan oil, rice germ oil, baobab oil, manetti oil, manila seed oil, yangu seed oil, rambutan oil, buckthorn oil, monoi de tahiti, tigernut oil, inca inchi oil, avocado oil, cottonseed oil, cupuacu butter, cashew oil, safflower oil, peanut oil, jojoba oil, chamomile oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and wheatgerm oil are particularly preferred, in particular (−)-α-bisabolol, hydrogenated jojoba oil and/or coconut oil. A particularly preferred nourishing constituent comprises the cosmetic agent D-panthenol. The advantage of these nourishing oils or nourishing constituents lies in the fact that they originate to the greatest possible extent from natural sources and are thus energy- and resource-friendly raw materials. in accordance with a preferred embodiment, the cosmetic agent can contain from about 0.0005 to about 3 wt %, preferably from about 0.001 to about 2 wt %, and particularly preferably from about 0.05 to about 1 wt % of the sum of nourishing oils and nourishing constituents, in relation to the total weight.

In further preferred embodiments of the present disclosure the cosmetic agent may also comprise preservatives, fragrance and optionally further additives.

Cosmetic agents that are very particularly preferred as contemplated herein comprise at least one of the following embodiments A) to D):

### A)

A cosmetic agent for temporarily shaping keratin fibres, in particular human hair, in a cosmetic carrier comprising a film former and a natural thickener, wherein the natural thickener comprises carrageenan and/or salts thereof, and the film former comprises a hydroxypropyl group-modified starch compound, and wherein the cosmetic agent is free from a fully synthetic film former.

### B)

A cosmetic agent for temporarily shaping keratin fibres, in particular human hair, in a cosmetic carrier comprising a film former and a natural thickener, wherein the cosmetic agent contains from about 70 to about 98 wt %, preferably from about 75 to about 95 wt %, more preferably from about 80 to about 90 wt % of water; from about 1.5 to about 20 wt %, preferably from about 2 to about 15 wt %, more preferably from about 3 to about 10 wt % of hydroxypropyl group-modified starch compound as film former; and from about 0.5 to about 10 wt %, preferably from about 1 to about 7 wt %, more preferably from about 2 to about 5 wt % of carrageenan as thickener, in each case in relation to the total weight of the cosmetic agent.

### C)

A cosmetic agent for temporarily shaping keratin fibres, in particular human hair, in a cosmetic carrier comprising a film former and a natural thickener, wherein the cosmetic agent contains from about 70 to about 98 wt %, preferably from about 75 to about 95 wt %, more preferably from about 80 to about 90 wt % of water; from about 1.5 to about 20 wt %, preferably from about 2 to about 15 wt %, more preferably from about 3 to about 10 wt % of hydroxypropyl group-modified starch compound; and from about 0.5 to about 10 wt %, preferably from about 1 to about 7 wt %, more preferably from about 2 to about 5 wt % of carrageenan, in each case in relation to the total weight of the cosmetic agent, wherein the cosmetic agent also comprises a solubiliser, which is preferably castor oil and/or an alkoxylated castor oil, which more preferably is a castor oil alkoxylated with from about 20 to about 80, even more preferably from about 30 to about 50 ethylene oxide groups, in particular a castor oil alkoxylated with about 40 ethylene oxide groups.

### D)

A cosmetic agent for temporarily shaping keratin fibres, in particular human hair, in a cosmetic carrier comprising a film former and a natural thickener, wherein the cosmetic agent contains from about 70 to about 98 wt %, preferably from about 75 to about 95 wt %, more preferably from about 80 to about 90 wt % of water; from about 1.5 to about 20 wt %, preferably from about 2 to about 15 wt %, more preferably from about 3 to about 10 wt % of hydroxypropyl group-modified starch compound; and from about 0.5 to about 10 wt %, preferably from about 1 to about 7 wt %, more preferably from about 2 to about 5 wt % of carrageenan, in each case in relation to the total weight of the cosmetic agent, wherein the cosmetic agent also comprises a solubiliser, which is preferably castor oil and/or an alkoxylated castor oil, which more preferably is a castor oil alkoxylated with from about 20 to about 80, even more preferably from about 30 to about 50 ethylene oxide groups, in particular a castor oil alkoxylated with about 40 ethylene oxide groups, and wherein the base of the hydroxypropyl group-modified starch compound is a starch of plant origin, which preferably is obtained from potatoes and/or tapioca.

A second subject of the present disclosure is the use of the cosmetic agent of the first subject of the present disclosure for nourishing keratin fibres, in particular human hair, for shaping and/or fixing the form of keratin fibres, in particular human hair, and/or for styling a hairstyle from human hair.

Features relating to preferred embodiments of the first subject of the present disclosure and described above only in that regard of course also apply, accordingly, as features of preferred embodiments for the second subject.

The following examples are intended to explain the subject matter of the present disclosure without limiting it in any way.

### Examples

The following formulations as contemplated herein were produced as hair gel:

| | E1* | E2* | E3* |
|---|---|---|---|
| Satiagel VPC 512 ¹⁾ | 2.0 | 4.0 | 6.0 |
| Euxyl PE 9010 ²⁾ | 1.0 | 1.0 | 1.0 |
| PGE 12 ³⁾ | 10.0 | 8.0 | 6.0 |
| Citric acid Monohydrate | 0.7 | 0.7 | 0.7 |
| (Regular) | | | |
| Parfum Taftic HU MOD 1 MA | 0.2 | 0.2 | 0.2 |
| PEG-40 Hydrogenated Castor | 0.5 | 0.5 | 0.5 |
| Oil (INCI) | | | |
| Water | to 100 | to 100 | to 100 |

*stated values in wt %
¹⁾ carrageenan
²⁾ agent comprising phenoxyethanol
³⁾ potato starch, propylene oxide content: 12 wt % propylene oxide

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic agent for temporarily reshaping keratin fibres, the cosmetic agent comprising, a cosmetic carrier, a film former, and a natural thickener, wherein the natural thickener comprises carrageenan and/or salts thereof, and wherein the film former comprises a hydroxypropyl group-modified starch compound.

2. The cosmetic agent according to claim 1, wherein the cosmetic agent is free from a fully synthetic film former.

3. The cosmetic agent according to claim 1, wherein a basis of the hydroxypropyl group-modified starch compound is a starch of plant origin, wherein the starch of plant origin is obtained from potatoes, maize, rice, peas, acorns, chestnuts, barley, wheat, bananas, millet, sorghum, oats, barley, rye, beans, sweet potato, arrowroot, cassava, tapioca and/or sago.

4. The cosmetic agent according to claim 1, wherein a weight ratio of the film former to the natural thickener is from about 40 to 1 to about 1 to 2.

5. The cosmetic agent according to claim 1, wherein the cosmetic carrier comprises water.

6. The cosmetic agent according to claim 5, wherein the cosmetic agent comprises
a) from about 70 to about 98 wt % of water,
b) from about 1.5 to about 20 wt % of the hydroxypropyl group-modified starch compound, and
c) from about 0.5 to about 10 wt % of the natural thickener, wherein the natural thickener is carrageenan,
in each case in relation to a total weight of the cosmetic agent.

7. The cosmetic agent according to claim 1, wherein the cosmetic agent further comprises a solubiliser.

8. The cosmetic agent according to claim 1, wherein the cosmetic agent also comprises a solubiliser, in an amount of from about 0.05 to about 2 wt %, in relation to a total weight of the cosmetic agent.

9. The cosmetic agent according to claim 1, wherein the cosmetic agent is transparent.

10. The cosmetic agent according to claim 3, wherein the starch of plant origin obtained from potatoes and/or tapioca.

11. The cosmetic agent according to claim 4, wherein the weight ratio of the film former to the natural thickener is from about 20 to 1 to about 1 to 1.

12. The cosmetic agent according to claim 11, wherein the weight ratio of the film former to the natural thickener is from about 15 to 1 to about 2 to 1.

13. The cosmetic agent according to claim 12, wherein the weight ratio of the film former to the natural thickener is about 5 to 1.

14. The cosmetic agent according to claim 5, wherein the water is acidified with a divalent or trivalent organic acid.

15. The cosmetic agent according to claim 14, wherein the divalent or trivalent organic acid is citric acid.

16. The cosmetic agent according to claim 6, wherein the cosmetic agent comprises
a) from about 75 to about 95 wt % of water,
b) from about 2 to about 15 wt % of the hydroxypropyl group-modified starch compound, and
c) from about 1 to about 7 wt % of the natural thickener, in each case in relation to the total weight of the cosmetic agent.

17. The cosmetic agent according to claim 7, wherein the solubiliser is castor oil and/or an alkoxylated castor oil.

18. The cosmetic agent according to claim 7, wherein the solubiliser is castor oil alkoxylated with from about 20 to about 80 ethylene oxide groups.

19. The cosmetic agent according to claim 7, wherein the solubiliser is castor oil alkoxylated with about 40 ethylene oxide groups.

20. The cosmetic agent according to claim 8, wherein the solubiliser is present in the amount of from about 0.1 to about 1 wt %, in relation to the total weight of the cosmetic agent.
